(19) United States
(12) Patent Application Publication
(10) Pub. No.: US 2012/0265452 A1
(43) Pub. Date: Oct. 18, 2012

(54) METHOD OF MONITORING GAS SUPPLY DURING A CRYOSURGICAL PROCEDURE

(75) Inventors: Satish Ramadhyani, Minneapolis, MN (US); Luan Thien Chan, Coon Rapids, MN (US); Jason W. Sprain, Shoreview, MN (US); Timothy J. Davis, Coon Rapids, MN (US); William M. Jacqmein, Woodbury, MN (US)

(73) Assignee: GALIL MEDICAL INC., Arden Hills, MN (US)

(21) Appl. No.: 13/086,977

(22) Filed: Apr. 14, 2011

(51) Int. Cl. G01F 1/36; A61B 18/02

(52) U.S. Cl. 702/47; 606/20

(57) ABSTRACT

The invention is a method for monitoring an amount of gas available in a cryosurgical system used for conducting a cryosurgical procedure. Based on information pertaining to a planned cryosurgical procedure, cryoprobe-specific information for each cryoprobe connected to the cryosurgical system, the specified volumetric capacity of a cryogas source, and measured pressure of the gas in the cryogas source, the method determines the mass flow rates of gas flowing through each cryoprobe, the total mass flow rate of the gas through the cryosurgical system, and the mass of the gas in the cryogas source at standard atmospheric conditions. The amount of time available for completing the cryosurgical procedure before the cryogas source is expended is determined as the arithmetic ratio of the mass of the gas in the cryogas source to the total mass flow rate of the gas through the cryosurgical system.

### TECHNICAL FIELD

The present invention relates to a method of monitoring the amount of cryogas being used during a cryosurgical procedure and enunciating the amount of time available for completing the procedure.

### BACKGROUND

Cryosurgical systems according to the prior art comprise one or more cryoprobes connected to a cryogas supply module which includes one or more cryogas sources and a controller. The controller is typically designed to receive control commands from a surgeon and, following those commands, to control valves governing delivery of cryogas from the cryogas sources to the connected cryoprobes. In this manner a surgeon, by commanding actions of the controller, controls delivery of cryogas to the cryoprobes, thereby controlling cooling and heating of those cryoprobes and the tissue in their vicinity.

Cryoprobes are well known devices used for therapeutic freezing and thawing of target tissue such as tumor. One class of cryoprobes utilizes the Joule-Thomson effect to produce cooling or heating. In these probes, a gas is passed from a first region of the device, where it is held under higher pressure, to a second region of the device, wherein it is enabled to expand to a lower pressure. This expansion, and the associated Joule-Thomson effect may occur in a simple conduit such as a capillary tube, or it may occur in an orifice, generally referred to as a Joule-Thomson orifice, through which gas passes from a first, higher pressure, region of the device to a second, lower pressure, region of the device. In some embodiments, a cryoprobe further includes a heat exchanger that is used to pre-cool gases within a first region of the device, prior to their expansion into a second region of the device. Effective operation of the cryoprobes depends on the availability of the cryogases at their specified pressures. Too low a pressure may result in inadequate cooling or heating performance.

Generally, the cryogas sources are separate gas tanks containing a high-pressure cooling gas and a high-pressure heating gas. The term “cooling gas,” as is well known in the art, refers to a gas which, at room temperature or colder, has the property of becoming colder when it is permitted to expand from a region of higher pressure into a region of lower pressure and may to some extent liquefy creating a pool of liquefied gas. Examples of “cooling gases” include argon, nitrogen, air, krypton, CO₂, CF₄, xenon, and various other gases. In a cryoprobe, the cooling gas is typically permitted to expand within the tip at the distal end of the cryoprobe whereat the expansion of the gas results in temperatures at or below those necessary for cryoablating a tissue in the vicinity of the tip of the cryoprobe. Typically, argon is used as the cooling gas for cooling the cryoprobes to sufficiently low temperatures for cryoablating the tissue in the vicinity of the tips of the cryoprobes.

The term “heating gas,” as is well known in the art, refers to a gas which, at room temperature or warmer, has the property of becoming hotter when it is permitted to expand from a region of higher pressure into a region of lower pressure. Helium is an example of a “heating gases.” In a cryoprobe, the heating gas is typically permitted to expand within the tip at the distal end of the cryoprobe whereat the expansion of the gas results in temperatures at or above those necessary for thawing a cryoablated tissue. Typically, helium is used as the heating gas for heating the cryoprobes to thaw the tissue in the vicinity of the tips of the cryoprobes for the purpose of un-freezing the cryoprobes from the cryoablated tissue.

Generally, before starting a cryosurgical procedure, the surgeon pre-plans a regimen that will be followed and selects the number and types of cryoprobes that will be used during the procedure. This planning process typically includes information such as the duration of time the cryoprobes will be operated in each of the cooling and heating modes, and the individually rated cooling and heating capacities of each cryoprobe. This information is then used to determine the total amount of cooling and heating gases that will be required for completing the planned procedure. Since fully-charged gas tanks or tanks with known pressure and size are installed before starting a cryosurgical procedure, the mass of both the cooling and heating gases in their respective tanks is known beforehand. Accordingly, before starting the procedure, the surgeon is able to determine whether or not a procedure can be completed without the need to replace one or more gas tanks.

However, during a cryosurgical procedure, several operating parameters can effect the actual amount of cooling and heating gases used and the depletion thereof from the gas tanks. For instance, the surgeon may determine it necessary to increase or decrease the duration of time a cryoprobe is operating in the heating or cooling mode. In other instances the surgeon may find it necessary to use more or fewer cryoprobes for heating or cooling purposes than initially anticipated during the pre-planning phase before the procedure. It is also possible that the gas tanks installed before starting the procedure were not full-charged. Under each of these exemplary situations, and unbeknownst to the surgeon, there then arises the possibility that the gas tanks may not contain a sufficient quantity of heating and/or cooling gas to permit completion of the cryosurgical procedure underway.

Accordingly, there exists a need for monitoring the amount of heating and cooling gases used during a cryosurgical procedure, and for providing the surgeon an estimate of the amount of time available before depletion of the gases from their respective tanks.

### SUMMARY

An embodiment of the invention comprises a method for monitoring an amount of gas available in a cryosurgical system used for conducting a cryosurgical procedure. Based on information pertaining to a planned cryosurgical procedure provided to a controller and cryoprobe-specific information provided to the controller for each cryoprobe connected to the controller, the controller computes the mass flow rates of gas flowing through each cryoprobe connected to the controller and computes the total mass flow rate of the gas through the cryosurgical system. Measurements of the pressure of the gas in a cryogas source and the specified volumetric capacity of the cryogas source are used by the controller to compute the mass of the gas in the cryogas source. Then, the amount of time available for completing the cryosurgical procedure before the cryogas source is expended is computed as the arithmetic ratio of the mass of the gas in the cryogas source at standard atmospheric conditions to the total mass flow rate of the gas through the cryosurgical system. The computed amount of time is then enunciated in a visual and/or audible form.

An embodiment of the invention comprises a system for monitoring an amount of gas available in a cryosurgical system used for conducting a cryosurgical procedure. Based on information pertaining to a planned cryosurgical procedure provided to a controller and cryoprobe-specific information provided to the controller for each cryoprobe connected to the controller, the system determines the mass flow rates of gas flowing through each cryoprobe connected to the controller and determines the total mass flow rate of the gas through the cryosurgical system. Measurements of the pressure of the gas in a cryogas source and the specified volumetric capacity of the cryogas source are used to determine the mass of the gas in the cryogas source at standard atmospheric conditions. Then, the amount of time available for completing the cryosurgical procedure before the cryogas source is expended is determined as the arithmetic ratio of the mass of the gas in the cryogas source at standard atmospheric conditions to the total mass flow rate of the gas through the cryosurgical system. The amount of time is then enunciated in a visual and/or audible form.

### BRIEF DESCRIPTION OF THE DRAWINGS

FIG. 1A is a schematic of a cryosurgical system comprising a cryoprobe having an embedded electronic module in accordance with an embodiment of the invention;

FIG. 1B is a block diagram of an embodiment of the electronic module of FIG. 1A;

FIG. 2 is a schematic of a cryosurgical system in accordance with an alternate embodiment of the invention;

FIG. 3 is a schematic of a cryosurgical system in accordance with another embodiment of the invention; and

FIG. 4 is a flowchart of a method for monitoring the amount of gases used during a cryosurgical procedure and for estimating the amount of time before depletion of the gas tanks.

### DETAILED DESCRIPTION

While multiple embodiments of the instant invention are disclosed, alternate embodiments may become apparent to those skilled in the art. The following detailed description describes only illustrative embodiments of the invention with reference to the accompanying drawings. It should be clearly understood that there is no intent, implied or otherwise, to limit the invention in any form or manner to that described herein. As such, all alternative embodiments are considered as falling within the spirit, scope and intent of the instant invention.

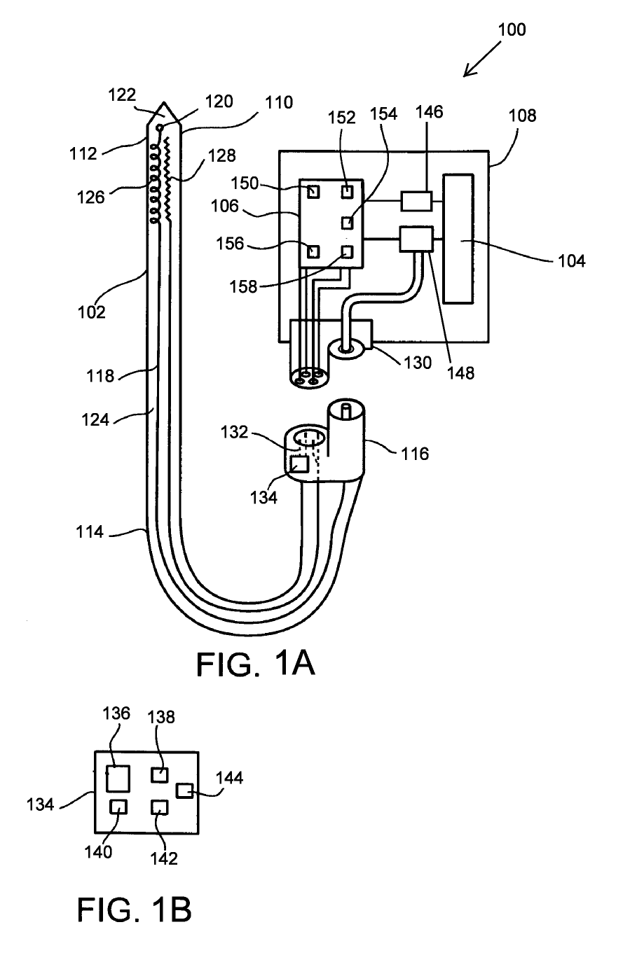

FIG. 1A illustrates cryosurgical system 100 comprising cryoprobe 102, cryogas source 104 for supplying a cryogas to cryoprobe 102, and controller 106 for controlling delivery of cryogas from cryogas source 104 to cryoprobe 102. In this non-limiting exemplary embodiment cryogas source 104 and controller 106 are shown housed in common cabinet 108. In alternate embodiments, cryogas source 104 is located external to and in fluid communication with cabinet 108. Cryoprobe 102 comprises distal portion 110 including treatment head 112 coolable by delivery thereto of a cryogas, flexible hose portion 114 and connector 116 on a proximal portion of cryoprobe 102. Cryogas supply conduit 118 supplies a cryogas (high-pressure cooling gas such as argon, or another cryogas) to Joule-Thomson orifice 120 in expansion chamber 122 in treatment head 112. Cryogas exhaust conduit 124 carries the expanded gas away from head 112 and back to connector 116. Heat exchanger 126 positioned in or near head 112 provides pre-cooling of high-pressure gas approaching head 112. However, it is to be understood that although Joule-Thomson cooling is presented in this exemplary embodiment, cooling by evaporation of a liquefied cryogas, or any other form of cooling, falls within the scope of the present invention.

In some embodiments of the invention, it is desirable to heat head 112 to facilitate disengagement of cryoprobe 102 after cryoablating a tissue, or for other purposes. As illustrated in this exemplary embodiment, optional electrical heater 128 is integrated with heat-exchanger 126 to provide heating of head 112. In other embodiments, optional electrical heater 128 is positioned elsewhere in cryoprobe 102 to provide heating of head 112. In yet other embodiments, controller 106 is configured for regulating a flow of electrical current to electrical heating element 128 within cryoprobe 102. In some embodiments, head 112 is heated using a heating gas such as high-pressure helium supplied by controller 106 and cryogas source 104. In alternate embodiments of the invention, head 112 is not heated.

Connector 116 on a proximal portion of cryoprobe 102 is used for connecting cryoprobe 102 to socket 130 on cabinet 108 for providing gas connection to cryogas source 104 and electrical/electronic connection to controller 106. In this exemplary embodiment, connector 116 is shown comprising power and data links 132, which may be a combined power and data link, and electronic module 134 embedded within connector 116. Power and data links 132 enable communication between module 134 and controller 106 through connector 116 and socket 130. While module 134 is shown as embedded within connector 116, it is should be understood that module 134 may be positioned anywhere in or on any part of cryoprobe 102, according to convenience of manufacture and/or convenience of use.

Attention is now drawn to FIG. 1B, which presents additional details of electronic module 134. In accordance with an embodiment of the present invention, module 134 comprises read/write memory 136 and/or read-only memory 138, communication interface 140, processor 142 and/or additional electronic components 144 (e.g. sensors, timer, analog/digital converters, etc.). Communication interface 140 provides a data transfer path between module 134 and controller 106.

Referring back to FIG. 1A, controller 106 comprises pressure transducers 146 and servo-controlled valves 148. Pressure transducers 146 measure the pressure of the cryogas in cryogas source 104; and servo-controlled valves 148 regulate, in a manner well known in the art, the flow of cryogas from cryogas source 104 to cryoprobe 102. In some embodiments controller 106 is programmed to regulate the flow of cryogas from cryogas source 104 to cryoprobe 102 in response to information received from module 134. Typically, the cryogas is a high-pressure cooling gas such as argon. In alternate embodiments, the cryogas is a liquefied gas operable to cool head 112 by evaporation. In other embodiments, source 104 contains a high-pressure heating gas such as helium for heating portions of cryoprobe 102.

As shown, controller 106 comprises memory 150, processor 152, user interface 154, and communications module 156. User interface 154 includes an input device such as a key board or a touch screen display, and an output device such as display. Various other input and output devices used as user interfaces, as are well known in the art, are also contemplated as alternate embodiments of the instant invention. In accordance with an embodiment of the invention, user interface 154 is used by a surgeon to provide operational and control instructions to controller 106.

In an embodiment of the invention, controller 106 is programmed to calculate and issue commands in response to information received from module 134. In an alternate embodiment, controller 106 is programmed to calculate and issue commands in response to information received from one or more sensors within cryoprobe 102 and/or sensors connected to controller 106 and/or sensors communicating with controller 106. In other embodiments, controller 106 is programmed to calculate and issue commands in response to commands issued by an operator. In yet other embodiments, controller 106 is programmed to calculate and issue commands in response to communications from a remote source (e.g. a network, the Internet, etc.) received through communications module 156.

Controller 106 is operable to read information from memories 136/138 of module 134 and optionally is operable to write information to memory 136 of module 134. Memories 136/138 contain information written during manufacture and/or factory calibration which are accessible to controller 106. Such information includes, but is not limited to, a unique identity code for each cryoprobe 102, cryoprobe type, cryoprobe specifications, test results, etc. Such cryoprobe specific information, readable by controller 106 during power-up (e.g. at the time of initial connection between cryoprobe 102 and controller 106) or at any other time, enables controller 102 determine cryoprobe specific operating parameters in view of a specific treatment plan. For example, the actual gas throughput of individual cryoprobes under identical cryogas pressure conditions typically varies somewhat. Accordingly, operating characteristics (e.g. cooling capacity) of individual cryoprobes from testing under standard conditions is encoded in memories 136/138 of each individual cryoprobe and subsequently used by controller 106 to determine optimal operating parameters (e.g. length of timed cooling operations). The use of such information provides a more accurately determinable cooling effect than that determinable merely according to theoretical cooling capacities or other characteristics specified only by their intended operating and manufacturing parameters. In alternate embodiments wherein cryoprobe 102 does not include module 134, the surgeon or an operator enters the identification information for cryoprobe 102 via user interface 154, and all necessary cryoprobe specific information is obtained from a configuration file in controller 106.

Controller 106 monitors, records and reports individual and collective cryoprobe usage statistics and limits or otherwise regulates cryoprobe re-use for commercial purposes and/or to enforce safety standards or for other clinical purposes. Testing status, measured operating statistics, activation history, and other cryoprobe specific information is usable to enable/disable use of individual cryoprobes 102. Controller 106 uses communications module 156 for communicating with a remote server, such as a server accessible through the Internet or by other communication means and operated by a manufacturer of cryosurgical system 100 or by a commercial intermediary such as a local supplier of cryosurgical system 100. Such communications is used to report cryoprobe usage patterns, to request and receive authorization for an operation, for inventory management, or for other purposes.

The capabilities mentioned in the preceding paragraph and elsewhere herein constitute a potential advantage of cryoprobe 102 and cryosurgical system 100 over prior art cryoprobes and cryosurgical systems. For example, some cryoprobe manufacturers instruct users to test cryoprobes prior to use, and to avoid excessive re-use, and users may even undertake an obligation to quantitatively limit cryoprobe re-use, yet prior art cryosurgical systems provided no means for verifying such user behavior nor for enforcing these limitations. As shown above, means for such verification and enforcement may be provided by cryosurgical system 100. Cryosurgical system 100 is optionally operable to ensure that only cryoprobes manufactured to be compatible with controller 106 are connected to and used with controller 106 during a surgical procedure.

It is noted that optional electronic components 144 and 158 are installed in module 134 and controller 106, respectively, to provide additional functionality. For example, components 144/150 comprise one or more sensors, timers, analog/digital converters, etc. Such components can be used, for example, as part of a temperature-reporting system wherein an ammeter or a voltmeter or a Wheatstone bridge is used to assess the temperature of a resistive heater as a function of the heater's electrical characteristics. Other forms of temperature sensors, pressure sensors, flow meters, or other sensors can also be interfaced through module 134 and/or controller 106. In some embodiments, components 144 and 158 comprise radio frequency communications devices or other communications devices enabling wireless communication between two or more of module 134, controller 106, a remote server, a network, the Internet, etc.

Although not explicitly illustrated, cabinet 108 is configured for enabling simultaneous connection of a plurality of cryoprobes 102, and controller 106 is configured for enabling simultaneous control and use of a plurality of cryoprobes 102. For simplicity, FIG. 1A shows only one such connection, viz., for connecting connector 116 on a proximal end of cryoprobe 102 to socket 130 on controller 106. Furthermore, and again for simplicity, FIG. 1A shows only one servo-controlled valve 148 for regulating, in a manner well known in the art, the flow of cryogas from cryogas source 104 to cryoprobe 102. As explained above, controller 106 verifies the identity and type of all cryoprobes 102 connected to controller 106 by communicating with electronic module 134 on each cryoprobe 102 connected to controller 106. Additionally, all cryoprobe-specific information encoded in electronic module 134 is accessible by controller 106. Accordingly, by taking into consideration all probe-specific information, controller 106 modifies the operating parameters of each connected cryoprobe 102. These capabilities enable the user, via controller 106, to tailor parameters such as cryogen flow times to one or more cryoprobes 102, thereby facilitating simultaneous use of a plurality of differing types of cryoprobes with the same controller 106 during an entire surgical procedure. Verification that cryoprobes 102 actually connected to controller 106 correspond to those whose connection was planned or intended is an additional feature provided by system 100.

An additional optional use of the cryosurgical system described herein above is to facilitate the use of cryoprobes of differing capacities simultaneously or sequentially with a common controller 106. Since each cryoprobe 102 is configured to supply self-descriptive information, controller 106 can be programmed to adapt its operational parameters to each cryoprobe individually, thus enabling a mixture of a plurality of cryoprobes with differing cooling and/or heating capacities or other differing operational characteristics and yet easily cause each cryoprobe to conform to a pre-determined common surgical plan (e.g. a planned ice-ball shape and size). As such, it is also possible to determine whether the characteristics of cryoprobes 102 actually connected to controller 106 correspond to cryoprobe characteristics called for in a surgical plan, thereby assuring that correctly characterized cryoprobes are inserted and used.

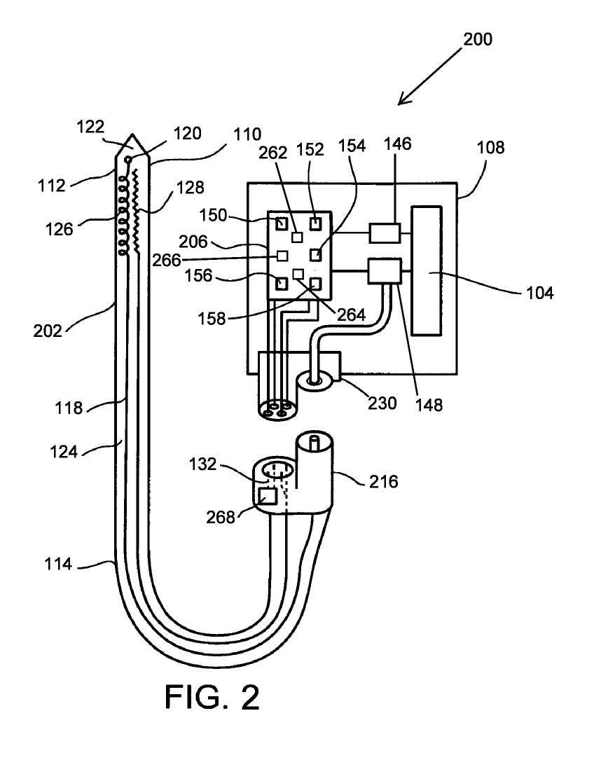

An alternate embodiment of cryosurgical system 100 described herein above with reference to FIGS. 1A and 1B is illustrated in FIG. 2 as cryosurgical system 200, wherein like elements are represented by like numerals. Accordingly, and in the interest of brevity, the following description in reference to FIG. 2 focuses only on those elements of cryosurgical system 200 that are different from cryosurgical system 100.

As with cryosurgical system 100, cryosurgical system 200 comprises cryoprobe 202, cryogas source 104 for supplying a cryogas to cryoprobe 202, and controller 206 for controlling delivery of cryogas from cryogas source 104 to cryoprobe 202.

In an embodiment of the invention, controller 206 includes data source 262 for obtaining cryoprobe specific information from a database within controller 206. In alternate embodiments, data source 262 is an interface for receiving cryoprobe specific information over a network or over the Internet or via wireless communication.

Controller 206 further comprises query module 264 for transmitting one or more query signals when one or more cryoprobes 202 are first connected to controller 206 or at any other time. In an embodiment of the invention, query module 264 functions to formulate, based on information received from data source 262, a query signal for transmission to cryoprobe 202. In an alternate embodiment, query module 264 transmits a series of query signals to cryoprobe 202 based on information known to controller 206 about one or more cryoprobes 202.

Controller 206 further comprises identifier module 266, for receiving from cryoprobe 202 a response to a query signal transmitted by query module 264, and for analyzing that response signal to determine if it is possible, based on that signal, to establish a unique cryoprobe-specific identity code for cryoprobe 202.

In an embodiment of the invention, connector 216 on a proximal portion of cryoprobe 202 includes response module 268 in the form of an electronic circuit. In an alternate embodiment, response module 268 is an embedded radio-frequency (RF) tag. Response module 268 is operable to recognize when a received query signal, transmitted by query module 264, possesses a predetermined characteristic, and to emit a characteristic response, which can be an encoded signal or a simple signal, when a query signal having said predetermined characteristic is recognized. In the simple embodiment mentioned above, wherein query signals are unique cryoprobe-specific identity codes, response module 268 simply tests an incoming signal to determine whether the incoming signal is recognized as its own unique cryoprobe-specific identity code. If it is, response module 268 transmits a “yes” response, whereby the cryoprobe is identified and the query process terminates. If it is not, response module 268 transmits a “no” response or does not transmit anything. In the event of a “no” or no response, query module 264 then transmits other queries based on information about other cryoprobes in its data list (obtained from data source 262 or any other source), cycling through its list of known cryoprobes until a match is found. In an alternate embodiment, query module 264 transmits a real-time date and asks for a response from cryoprobes whose expiration date is prior to, or alternatively later than, the transmitted real-time date. Response module 268 comprising a memory containing an expiration date can recognize a query signal encoding a real-time date, and appropriately transmit a “yes” or a “no” response.

At that point, controller 206 knows which of the cryoprobes 202 known to it is attached at the position to which the queries are sent. From then on, the various procedures and methods outlined above with respect to cryosurgical system 100 are undertaken. Information read from data source 262 and now associated with specific cryoprobes 202 connected to controller 206 can include information characterizing a usage history of such cryoprobes, data derived from an operational test of the cryoprobes, a type designation for the cryoprobes, a descriptive characterization of the cryoprobes, etc.

Although not explicitly illustrated, cabinet 108 is configured for enabling simultaneous connection of a plurality of cryoprobes 202, and controller 206 is configured for enabling simultaneous control and use of a plurality of cryoprobes 202. For simplicity, FIG. 2 shows only one such connection, viz., for connecting connector 216 on a proximal end of cryoprobe 202 to socket 230 on controller 206. Furthermore, and again for simplicity, FIG. 2 shows only one servo-controlled valve 148 for regulating, in a manner well known in the art, the flow of cryogas from cryogas source 104 to cryoprobe 202. As explained above, probe-specific information obtained from data source 262 is used by controller 206 to verify the identity and type of all cryoprobes 202 connected to controller 206. Verification that cryoprobes 202 actually connected to controller 206 correspond to those whose connection was planned or intended is an additional feature provided by system 200.

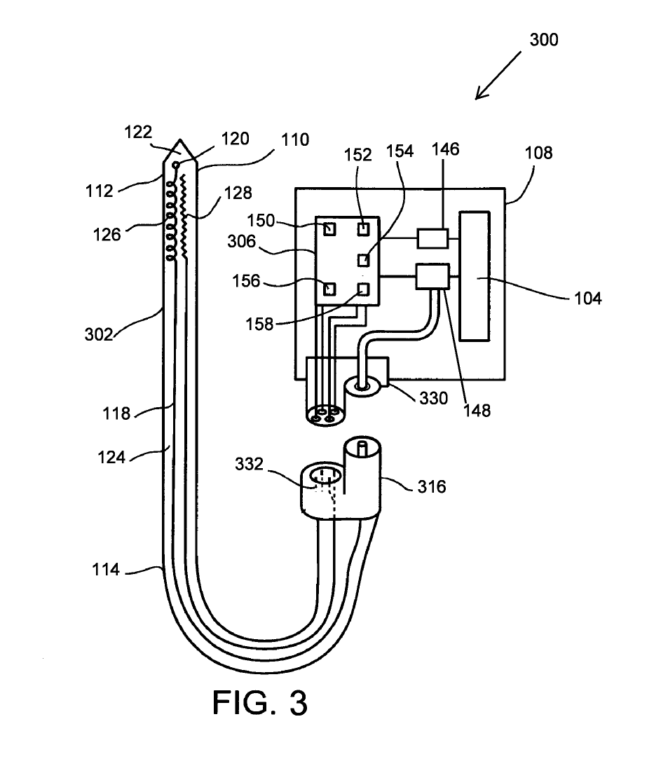

Another embodiment of cryosurgical systems 100 and 200 described herein above with reference to FIGS. 1A and 2 is illustrated in FIG. 3 as cryosurgical system 300, wherein like elements are represented by like numerals. Accordingly, and in the interest of brevity, the following description in reference to FIG. 3 focuses only on those elements of cryosurgical system 300 that are different from the embodiments of cryosurgical systems 100 and 200.

FIG. 3 illustrates cryosurgical system 300 comprising cryoprobe 302, cryogas source 104 for supplying a cryogas to cryoprobe 302, and controller 306 for controlling delivery of cryogas from cryogas source 104 to cryoprobe 302. Connector 316 on a proximal portion of cryoprobe 302 is used for connecting cryoprobe 302 to socket 330 on cabinet 108 for providing gas connection to cryogas source 104 and electrical connection to controller 306. In this embodiment, connector 316 includes power link 332 but no data link. As such, connector 316 does not include a communications link or a response module such as power and data links 132 in connector 116 or response module 268 in connector 216.

Additionally, in an embodiment of cryosurgical system 300, controller 306 does not include any other automated means for obtaining any cryoprobe identity codes and/or any other cryoprobe-specific information. For instance, controller 306 does not include a data source such as data source 262 in an embodiment of controller 206.

As described in the foregoing with reference to controllers 106 and 206, controller 306 in an embodiment of cryosurgical system 300 is programmed to calculate and issue commands and, in general, to operate cryosurgical system 300 in response to one or more of commands issued by an operator or in response to information received from one or more sensors connected to and/or communicating with controller 306 or in response to communications from a remote source (e.g. a network, the Internet, etc.). Accordingly, user interface 154 is used for providing all cryoprobe-specific information to controller 306 for every cryoprobe 302 connected to controller 306. In accordance with an embodiment of the invention, cryoprobe-specific information is provided to controller 306 in a sequential manner, i.e., as each cryoprobe 302 is placed within a tissue and connected to controller 306 via socket 330, the surgeon configures controller 306 to recognize the newly connected cryoprobe 302 and thereafter enters the cryoprobe-specific information in an associative manner.

User interface 154 includes an input device such as a key board or a touch screen display, and an output device such as display. Various other input and output devices used as user interfaces, as are well known in the art, are also contemplated as alternate embodiments of the instant invention. In accordance with an embodiment of the invention, user interface 154 is used by a surgeon to provide operational and control instructions to controller 306.

As described in the foregoing with reference to cryosurgical systems 100 and 200, the information provided to controller 306 through user interface 154 typically includes all or a subset of the information (e.g., the unique identity code of each cryoprobe 302, cryoprobe type, cryoprobe specifications, test results, actual gas throughput, etc.) provided to controller 106 via electronic module 134 or provided to controller 206 via data source 262.

Although not explicitly illustrated, cabinet 108 is configured for enabling simultaneous connection of a plurality of cryoprobes 302, and controller 306 is configured for enabling simultaneous control and use of a plurality of cryoprobes 302. For simplicity, FIG. 3 shows only one such connection, viz., for connecting connector 316 on a proximal end of cryoprobe 302 to socket 330 on controller 306. Furthermore, and again for simplicity, FIG. 3 shows only one servo-controlled valve 148 for regulating, in a manner well known in the art, the flow of cryogas from cryogas source 104 to cryoprobe 302.

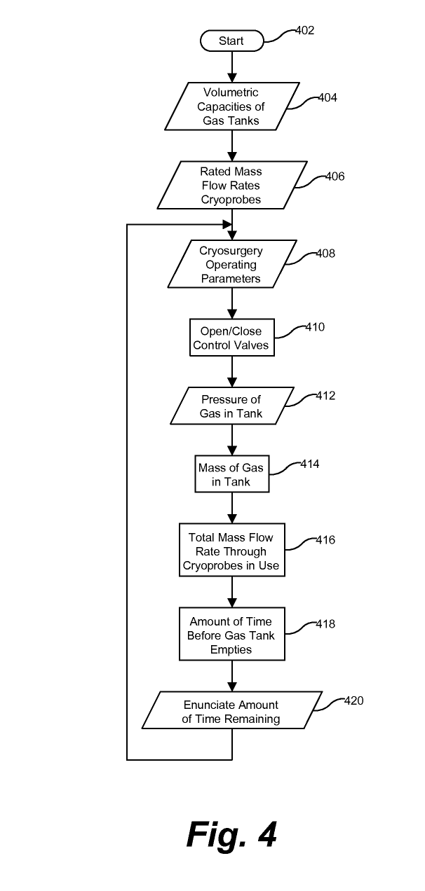

In accordance with an embodiment of the invention, FIG. 4 is a flowchart illustrating the steps associated with a method for monitoring the use of cryogas during a cryosurgical procedure and enunciating the amount of time available for completing the procedure before cryogas source 104 is expended. The method may be implemented as a stand-alone program running on controller 106/206/306 or it may be a subroutine or a sub-program executed under the control of one or more other programs running on controller 106/206/306. Starting at block 402 and using user interface 154, the surgeon is required, at block 404, to specify the volumetric capacities of cryogas source 104.

Next, at block 406, controller 106/206/306 receives the rated mass flow rate for each cryoprobe 102/202/302 as the surgeon positions each cryoprobe 102/202/302 into the tissue of a patient and connects it to controller 106/206/306 by connecting connectors 116/216/316 to socket 130/230/330 on cabinet 108. As described in the foregoing, controller 106 receives the rated mass flow rates for cryoprobes 102 from their individual electronic modules 134; controller 206 receives the rated mass flow rates for cryoprobes 202 from data source 262; and the rated mass flow rates for cryoprobes 302 are entered into controller 306 from user interface 154. In an alternate embodiment, such cryoprobe-specific information is retrieved by controller 106/206/306 through communications module 156.

After connecting cryoprobes 102/202/302 to controller 106/206/306, the surgeon, at block 408, uses user interface 154 to activate and deactivate (or enable and disable) cryoprobes 102/202/302 used during the cryosurgical procedure and to specify the operating parameters for the cryosurgical procedure. Typical operating parameters that can be effectuated by the surgeon at block 408 include the cryoprobe duty cycle which specifies the duration of time for which each individual servo-controlled valve 148 associated with each cryoprobe 102/202/302 remains in the open and closed positions. As used herein, a cryoprobe duty cycle is defined as the arithmetic ratio of the duration of time for which the individual control valve is in the open position to the total cycle time for that valve. Accordingly, setting a cryoprobe duty cycle to zero is essentially the same as deactivating (or disabling) the particular cryoprobe from further use until the duty cycle is reset to a non-zero value. Next, at block 410, controller 106/206/306 operates servo-controlled valves 148 in accordance with the operating parameters specified by the surgeon at block 408.

At block 412 controller 106/206/306 communicates with pressure transducer 146 for obtaining the pressure of the gas in cryogas source 104. Then, based on the volumetric capacity of the gas tank specified at block 404, the pressure measured at block 412 and using algorithms well known in the art, at block 414 controller 106/206/306 computes the mass of the gas in cryogas source 104 at standard atmospheric condition.

Next, at block 416, controller 106/206/306 first computes the mass flow rate of the gas flowing through each cryoprobe 102/202/302 connected to controller 106/206/306 as the arithmetic product of that cryprobe's duty cycle provided to controller 106/206/306 at block 408 and that cryoprobe's rated mass flow rate provided to controller 106/206/306 at block 406. Then, controller 106/206/306 computes the total mass flow rate of the gas flowing through cryosurgical system 100/200/300 as the arithmetic total of the mass flow rate of gas flowing through each cryoprobe 102/202/302 connected to controller 106/206/306. Now, the amount of time available before the gas tank is depleted is calculated at block 418 as the arithmetic ratio of the mass of gas remaining in the gas tank as determined at block 414 to the total mass flow rate of the gas being used by cryosurgical system 100/200/300 as determined at block 416.

Then, the amount of time is enunciated at block 420. In some embodiments of the invention, the enunciation is in the form of a display on user interface 154. In other embodiments, the enunciation is in the form of an audible and/or a visual alarm. In alternate embodiments, the frequency and manner of enunciation is a function of the amount of time available.

Various modifications and additions may be made to the exemplary embodiments presented hereinabove without departing from the spirit, scope and intent of the present invention. For example, while the disclosed embodiments refer to particular features, the scope of the instant invention is considered to also include embodiments having various combinations of features different from and/or in addition to those described hereinabove. Accordingly, the present invention embraces all such alternatives, modifications, and variations as within the spirit, scope and intent of the appended claims, including all equivalents thereof.

We claim:

1. A method for monitoring an amount of gas available in a cryosurgical system used for conducting a cryosurgical procedure, the method comprising:
receiving a volumetric capacity of a tank containing the gas;
receiving performance characteristics of a plurality of cryoprobes, wherein the performance characteristic includes at least a rated mass flow rate for the gas flowing through each cryoprobe;
receiving operating parameters for the cryosurgical system;
operating the cryosurgical system responsive to said operating parameters;
measuring a pressure of the gas in the tank;
computing a mass of the gas in the tank;
computing a mass flow rate of the gas flowing through each one of the plurality of cryoprobes;
computing a total mass flow rate of the gas flowing through the cryosurgical system;
computing an amount of time available for completing the cryosurgical procedure; and
enunciating the amount of time available.

2. The method of claim 1, further comprising
changing said operating parameters during said cryosurgical procedure;
receiving said changed operating parameters; and
operating the cryosurgical system responsive to said changed operating parameters.

3. The method of claim 2, wherein the operating parameters comprise a plurality of cryoprobe duty cycles for operating a valve associated with each one of the plurality of cryoprobes, and the method comprises operating the valve to regulate a flow of the gas flowing through the cryoprobe associated therewith.

4. The method of claim 3, wherein the mass flow rate of the gas flowing through each one of the plurality of cryoprobes is the arithmetic product of the cryoprobe's duty cycle and the rated mass flow rate for the gas flowing through the cryoprobe.

5. The method of claim 4, wherein the total mass flow rate of the gas flowing through the cryosurgical system is the arithmetic sum of the mass flow rate of the gas flowing through each one of the plurality of cryoprobes.

6. The method of claim 5, wherein the mass of the gas in the tank is computed at standard atmospheric conditions using the volumetric capacity of the tank and the measured pressure of the gas in the tank.

7. The method of claim 6, wherein the amount of time available for completing the cryosurgical procedure is computed as an arithmetic ratio of the mass of the gas in the tank to the total mass flow rate of the gas flowing through the cryosurgical system.

8. The method of claim 7, wherein the steps of receiving and the steps of enunciating comprise one or more communications module, the communications module selected from the group consisting of: a data card reader, a data source, a network, a server, the Internet, a user interface device, a smart card, a device for transmitting and/or receiving wireless signals.

9. The method of claim 8, wherein the step of enunciating comprises operating one or more audio and/or visual alarm.

10. The method of claim 9, further comprising enunciating a low pressure condition when the measured pressure of the gas in the tank is less than a specified threshold, wherein the step of enunciating comprises operating one or more audio and/or visual alarm.

11. A means for monitoring an amount of gas available in a cryosurgical system used for conducting a cryosurgical procedure, the means comprising:
means for receiving a volumetric capacity of a tank containing the gas;
means for receiving performance characteristics of a plurality of cryoprobes, wherein the performance characteristic includes at least a rated mass flow rate for the gas flowing through each cryoprobe;
means for specifying operating parameters for the cryosurgical system;
means for receiving said operating parameters for the cryosurgical system;
means for operating the cryosurgical system responsive to said operating parameters;
means for measuring a pressure of the gas in the tank;
means for computing
a mass of the gas in the tank;
a mass flow rate of the gas flowing through each one of the plurality of cryoprobes;
a total mass flow rate of the gas flowing through the cryosurgical system; and
an amount of time available for completing the cryosurgical procedure; and
means for enunciating the amount of time available.

12. The means of claim 11, further comprising
means for changing said operating parameters during said cryosurgical procedure;
means for receiving said changed operating parameters; and
means for operating the cryosurgical system responsive to said changed operating parameters.

13. The means of claim 12, wherein the means for operating the cryosurgical system comprises
means for receiving a plurality of cryoprobe duty cycles for operating a valve associated with each one of the plurality of cryoprobes; and
means for operating the valve operating to regulate a flow of the gas flowing through the cryoprobe associated with said valve.

14. The means of claim 13, wherein the means for receiving and the means for enunciating comprise one or more communications module, the communications module selected from the group consisting of: a data card reader, a data source, a network, a server, the Internet, a user interface device, a smart card, a device for transmitting and/or receiving wireless signals.

15. The means of claim 14, further comprising means for operating one or more audio and/or visual alarm.

16. The means of claim 15, further comprising means for enunciating a low pressure condition when the measured pressure of the gas in the tank is less than a specified threshold, wherein the means for enunciating comprises means for operating one or more audio and/or visual alarm.

17. A system for monitoring an amount of gas available in a cryosurgical system used for conducting a cryosurgical procedure, the system comprising:
a controller;
a plurality of cryoprobes in operational communication with the controller;
a plurality of valves in operational communication with the controller, wherein each valve is operated by the controller to regulate a flow of gas through the cryoprobe associated with said valve;
a user interface in operational communication with the controller for providing a volumetric capacity of a tank containing the gas;
a plurality of communications modules in operational communication with the controller, wherein the plurality of communications modules
facilitate providing performance characteristics of a plurality of cryoprobes, wherein the performance characteristic includes at least a rated mass flow rate for the gas flowing through each cryoprobe;
operate the cryosurgical system responsive to specified operating parameters; and
enunciate an amount of time available for completing the cryosurgical procedure;
a pressure measuring device for measuring a pressure of the gas in the tank, said pressure measuring device in operational communication with the controller; and
a computing device for
computing a mass of the gas in the tank;
computing a mass flow rate of the gas flowing through each one of the plurality of cryoprobes;
computing a total mass flow rate of the gas flowing through the cryosurgical system; and
computing an amount of time available for completing the cryosurgical procedure.

18. The system of claim 17, wherein said plurality of communications modules further facilitate
changing said performance characteristics of said plurality of cryoprobes during said cryosurgical procedure; and
operating the cryosurgical system responsive to said changed operating parameters.

19. The system of claim 18, comprising operating the valves to regulate the flow of gas through the cryoprobes in accordance with a specified duty cycle for each one of the plurality of cryoprobes.

20. The system of claim 19, wherein the mass flow rate of the gas flowing through each one of the plurality of cryoprobes is the arithmetic product of the cryoprobe's duty cycle and the rated mass flow rate for the gas flowing through the cryoprobe.

21. The system of claim 20, wherein the total mass flow rate of the gas flowing through the cryosurgical system is the arithmetic sum of the mass flow rate of the gas flowing through each one of the plurality of cryoprobes.

22. The system of claim 21, wherein the mass of the gas in the tank is computed at standard atmospheric conditions using the volumetric capacity of the tank and the measured pressure of the gas in the tank.

23. The system of claim 22, wherein the amount of time available for completing the cryosurgical procedure is computed as an arithmetic ratio of the mass of the gas in the tank to the total mass flow rate of the gas flowing through the cryosurgical system.

24. The system of claim 23, wherein the plurality of communications modules enunciate a low pressure condition when the measured pressure of the gas in the tank is less than a specified threshold.

25. The system of claim 24, wherein the plurality of communications modules are selected from the group consisting of: a data card reader, a data source, a network, a server, the Internet, a user interface device, a smart card, a device for transmitting and/or receiving wireless signals, an audible alarm, and a visual alarm.
